(19) United States
(12) Patent Application Publication
(10) Pub. No.: US 2013/0165699 A1
(43) Pub. Date: Jun. 27, 2013

(54) PROCESS FOR PURIFYING CRUDE GLYCEROL

(71) Applicant: Sergio Mastroianni, Lyon (FR)

(72) Inventor: Sergio Mastroianni, Lyon (FR)

(73) Assignee: RHODIA OPERATIONS, Aubervilliers (FR)

(21) Appl. No.: 13/822,234

(22) PCT Filed: Sep. 7, 2011
(86) PCT No.: PCT/EP2011/065437

§ 371 Date: Mar. 11, 2013

(30) Foreign Application Priority Data
Sep. 15, 2010 (FR) 1003665

(51) Int. Cl. C07C 29/86

(52) U.S. Cl. CPC C07C 29/86
USPC 568/869

(57) ABSTRACT

A process is described for purifying crude glycerol obtained from raw materials such as glycerol obtained during manufacture of biodiesel or glycerol obtained during conversions of fats or oils. Further described, is how the process dissolves glycerol in an organic solvent and forms an insoluble phase comprising the salts of the crude glycerol.

The present invention relates to a process for the purification of the crude glycerol obtained from starting materials such as the glycerol obtained during the manufacture of biodiesel or the glycerol obtained during the conversion of fats or oils. The invention is targeted in particular at dissolving the glycerol in an organic solvent and forming an insoluble phase comprising the salts of the crude glycerol.

### PRIOR ART

Glycerol, 1,2,3-propanetriol, is present in the combined form in plant and animal oils and fats. It is in particular present in the form of triglycerides combined with fatty acids, such as stearic acid, oleic acid, palmitic acid and lauric acid. The most widespread industrial process for obtaining glycerol from plant and animal oils and fats involves saponification reactions, high pressure hydrolysis reactions and transesterification reactions with alcohols, such as ethanol or methanol.

Glycerol is also a byproduct from biodiesel which is obtained generally by the transesterification of glycerides with short-chain alcohols, for example methanol or ethanol.

The transesterification reaction is catalyzed by an acid or a base, according to the characteristics of the oils and/or fats used. After the transesterification reaction, the resulting esters are separated from the excess reactants, from the catalyst and from the byproducts by a process comprising two stages. First, the glycerol is separated by settling or centrifuging and then the soaps, the catalyst residues and alcohol residues are removed by washing with water and sparging or use of magnesium silicate with filtration. The extensive production of biodiesel as alternative to fossil sources is accompanied by high production of glycerol obtained as byproduct.

Depending on the manufacturing processes, the crude glycerol obtained comprises impurities which involve numerous and complex treatment stages.

To this end, it is known in particular to purify the crude glycerol by distillation, the operation being carried out with specific conditions in order not to detrimentally affect the glycerol, which decomposes at temperatures of 170-180° C. and which can polymerize and generate impurities. Such a purification process is therefore not advantageous industrially.

There thus exists a number of complex techniques which have been developed in the past in order to purify glycerol while avoiding decompositions or other undesirable reactions.

For example, patent U.S. Pat. No. 4,655,879 describes a very laborious process for the purification of crude glycerol which involves a large number of stages in which the crude glycerol is first basified in the presence of air for oxidation and then distilled at high temperatures under reduced pressures. As the glycerol obtained exhibits an undesirable color, it is furthermore necessary to carry out an additional treatment with activated charcoal.

Patent U.S. Pat. No. 4,990,695 describes the purification of crude glycerol with a combination of operations, such as the adjustment of the pH within a range from 9 to 12, heating the medium at 100° C., microfiltration and subsequent ultrafiltration. The glycerol obtained is then distilled, optionally after a treatment with ion-exchange compounds.

The prior art also knows other complex methods, such as electrodeionization or treatment with phosphoric acid or sulfuric acid, which necessitate several restricting separation stages.

The development is thus desired of a simple and industrial process for the purification of glycerol from crude glycerol which is relatively inexpensive and which takes place under ordinary temperature and pressure conditions and which makes it possible to obtain purified glycerol having a quality appropriate for a certain number of applications, while avoiding the abovementioned disadvantages.

### INVENTION

It has now been demonstrated that it is possible to purify crude glycerol by a process which is simple to carry out, which is efficient and which furthermore does not detrimentally affect the glycerol or its color. This process consists in adding, to the crude glycerol, an amount of organic solvent which will dissolve the glycerol and thus form an insoluble phase composed of a heterogeneous mixture of salts and of a few organic compounds. This insoluble phase will subsequently be separated from the liquid medium and, finally, the solvent will be separated from the purified glycerol. There are numerous advantages to such a process. Specifically, this process makes possible excellent purification and separation of the glycerol, whatever the type of crude glycerol used. In particular, a purity of greater than 95%, preferably of greater than 97%, of glycerol containing very small amounts of residual salts is obtained, and the solvent used can be fully recycled.

The present invention thus relates to a process for the purification of crude glycerol comprising at least the following stages:

(a) bringing together crude glycerol and at least one organic solvent comprising from 1 to 10 carbon atoms and comprising at least one ketone, aldehyde, alcohol, acetal and/or ketal functional group, so as to form a liquid phase comprising the glycerol dissolved in the solvent or solvents and to also form an insoluble phase;

(b) separation of the insoluble phase and of the liquid phase;

(c) separation of the solvent and of the glycerol of the liquid phase; and

(d) recovery of the glycerol.

The insoluble phase is generally a heterogeneous dispersed phase in the predominant phase and exists in the form of a precipitate.

The process of the invention can be carried out continuously or batchwise. The stages mentioned can be carried out successively and in or not in succession to one another. Each of the stages of the process can be carried out continuously or batchwise. Preferably, the process according to the invention is carried out by the strict sequence of the stages a), b), c) and d) without carrying out an intermediate or additional stage. In particular and advantageously, said liquid phase comprising the dissolved glycerol formed during said stage a) is not subjected, in itself, to any other treatment than the separation of said stage c) during which the glycerol is separated from the solvent.

The crude glycerol is preferably obtained from renewable starting materials; in particular, the crude glycerol is obtained during the manufacture of biodiesel or is obtained during conversions of fats or oils, particularly animal or vegetable fats or oils. The crude glycerol is generally obtained by a saponification, transesterification and/or hydrolysis reaction on animal or vegetable fats or oils.

The crude glycerol generally comprises from 5 to 95% by weight of glycerol, in particular from 40 to 90% by weight of glycerol and more particularly from 60 to 90% by weight of glycerol. The crude glycerol also comprises inorganic salts, glycerides, water and other organic compounds.

The crude glycerol can optionally be treated for the process of the invention, in particular, for example, by adjustment of the pH, filtration or distillation. It is thus possible to filter the crude glycerol in order to remove insoluble organic materials and/or to distil it, generally at temperatures of between 100 and 120° C. at atmospheric pressure, in order to remove water and volatile compounds. It is also possible to evaporate a portion or all of the water present in the crude glycerol before the dissolution of the glycerol in the solvent.

Stage a) of the process according to the invention is targeted at dissolving the glycerol in the organic solvent and forming an insoluble phase comprising the salts of the crude glycerol.

One or more solvents can be used. The solvent according to the invention can be in particular a ketone, an alcohol, an aldehyde, an acetal and/or a ketal. Acetals are obtained by nucleophilic addition of an alcohol to an aldehyde in an acidic medium, followed by removal of water. Ketals are obtained by the same type of reaction carried out on ketones.

The ketones preferably used are acetone, cyclohexanone, methylcyclohexanone, cyclopentanone, methylcyclopentanone and methyl isobutyl ketone (MIBK). The aldehydes preferably used are formaldehyde, acetaldehyde and furfuraldehyde. The alcohols preferably used are ethanol, methanol and isopropanol. The ketals and acetals are preferably dioxolanes, such as 2,2-dimethyl-1,3-dioxolane-4-methanol (solketal), for example.

Preference is given in particular to a mixture of organic solvents, such as a mixture of alcohol and ketone, particularly a mixture of acetone and ethanol.

In stage a), use will not be made of catalyst capable of catalyzing a reaction between the glycerol and the organic solvent or solvents of the medium, in particular not of an esterification catalyst.

Stage a) can last between 2 minutes and 1 hour. It can be carried out at a temperature of between 10 and 100° C., in particular between 20 and 50° C. The pH during this stage can be between 6 and 12 and preferably between 7 and 12.

The ratio by weight of the crude glycerol to the solvent (crude glycerol/solvent) is in particular a function of the solubility of the glycerol in said solvent and is, for example, preferably between 1/1 and 1/50, more preferably between 1/1 and 1/25.

Stage b) is targeted at the separation of the precipitate obtained in stage a) from the liquid phase comprising the solvent and the dissolved glycerol. It is possible in particular to carry out a filtration, a separation by settling or a centrifuging.

Stage c) is targeted at the separation of the solvent and of the glycerol which is dissolved in the solvent. It is possible in particular to carry out an evaporation or a distillation in order to do this.

In stage c), it is also possible to carry out the separation of the water present in the crude glycerol.

The evaporation will consist in particular in converting the organic solvent or solvents to the gaseous state, so as to be able to recover the glycerol in the liquid state.

Use may be made of one or more distillation columns in carrying out the distillation. It is possible in particular to distil the various compounds on the same distillation column by varying the temperature and optionally the pressure; for example to distil the organic solvent, then an increase in the temperature in order to distil the glycerol. Use is usually made of temperatures between 60 and 190° C. and pressures between 2 and 1000 mbar.

The purified glycerol is subsequently obtained and can be used in various applications requiring a high degree of purity.

A specific language is used in the description so as to facilitate the understanding of the principle of the invention. Nevertheless, it should be understood that no limitation on the scope of the invention is envisaged by the use of this specific language. Modifications and improvements can in particular be envisaged by a person conversant with the technical field concerned on the basis of his own general knowledge.

The term “and/or” includes the meanings “and”, “or” and all the other possible combinations of the elements connected to this term.

Other details or advantages of the invention will become more clearly apparent in the light of the examples given below solely by way of indication.

### Experimental Part

### EXAMPLE 1

The crude glycerol available commercially exhibits the following composition: 79.3% by weight of glycerol, 15.8% by weight of water, 1.61% by weight of Na⁺and 2.56% by weight of Cl⁻.

120.2 g of crude glycerol are added at ambient temperature to 2464 g of acetone. The mixture is stirred for 15 minutes, until the glycerol has dissolved in the acetone and an insoluble phase has been formed. The liquid phase is filtered off using a PTFE filter with a pore diameter of 0.2 μm and then the solvent and the water are evaporated under vacuum at a temperature of 60° C. and a pressure of 0.3 bar absolute.

88.93 g of purified glycerol, comprising 97.5% by weight of glycerol, are then recovered.

### EXAMPLE 2

150.8 g of crude glycerol are added at ambient temperature to 91.3 g of ethanol and 1754.3 g of acetone. The mixture is stirred for 20 minutes, until the glycerol has dissolved in the acetone and an insoluble phase has been formed. The liquid phase is filtered off using a PTFE filter with a pore diameter of 0.2 μm and then the solvent is evaporated under vacuum at a temperature of 75° C. and a pressure of 0.3 bar absolute.

106.7 g of purified glycerol, comprising 98.3% by weight of glycerol, are then recovered.

1. A process for the purification of crude glycerol, the process comprising at least the following stages:
(a) bringing together crude glycerol and at least one organic solvent comprising 1 carbon atom to 10 carbon atoms and comprising at least one ketone, aldehyde, alcohol, acetal and/or ketal functional group, so as to form a liquid phase comprising the glycerol dissolved in the solvent or solvents and to also form an insoluble phase in the form of a precipitate;
(b) separation of the insoluble phase and of the liquid phase;
(c) separation of the solvent and of the glycerol of the liquid phase; and
(d) recovery of the glycerol.

2. The process as defined by claim 1, wherein the crude glycerol originates from a renewable starting material.

3. The process as defined by claim 1, wherein the crude glycerol is obtained during manufacture of biodiesel or during conversion of a fat or an oil.

4. The process as defined by claim 1, wherein the ketone is selected from the group consisting of an acetone, a cyclohexanone, a methylcyclohexanone, a cyclopentanone, a methylcyclopentanone and a methyl isobutyl ketone.

5. The process as defined by claim 1, wherein the aldehyde is selected from the group consisting of a formaldehyde, an acetaldehyde and a furfuraldehyde.

6. The process as defined by claim 1, wherein the alcohol is selected from the group consisting of an ethanol, a methanol and an isopropanol.

7. The process as defined by claim 1, wherein the ketal or the acetal is a dioxolane.

8. The process as defined by claim 1, wherein the ratio by weight of the crude glycerol to the solvent (crude glycerol/solvent) is from 1/1 to 1/50.

9. The process as defined by claim 1, wherein the separation of stage b) is carried out by filtration.

10. The process as defined by claim 1, wherein the separation of stage c) is carried out by evaporation.

11. The process as defined by claim 3, wherein the fat or oil is an animal or vegetable fat or oil.
